(19) United States
(12) Patent Application Publication
(10) Pub. No.: US 2023/0228117 A1
(43) Pub. Date: Jul. 20, 2023

(54) EASY SNAP-TOGETHER ELECTRIC POWER TRACK APPARATUS AND METHOD FOR EFFICIENTLY TRANSFERRING ELECTRIC POWER TO DOORS FOR ENERGIZING DOOR-ASSOCIATED ELECTRICAL DEVICES

(71) Applicant: Tom Chi, Wichita, KS (US)

(72) Inventor: Tom Chi, Wichita, KS (US)

(21) Appl. No.: 17/927,849

(22) PCT Filed: Jun. 3, 2021
(86) PCT No.: PCT/US2021/035764

§ 371 Date: Nov. 25, 2022

Related U.S. Application Data
(60) Provisional application No. 63/034,475, filed on Jun. 4, 2020.
(60) Provisional application No. 63/040,663, filed on Jun. 18, 2020.

(51) Int. Cl. E05B 1/00; H02G 3/04; H02G 3/06

(52) U.S. Cl. CPC E05B 1/0069; H02G 3/0437; H02G 3/0608

(57) ABSTRACT

A snap-together electric power track apparatus for efficiently transferring electric power to doors in order to energize door-associated electrical devices includes a door access device associated with a door wherein a user comes into contact with the door access device utilizing electrical energy, a power hinge for transmitting electrical power the door, and a device for transmitting electrical energy to electrically energize one or more door-associated electrical devices. Preferably, the device for transmitting electrical energy to electrically energize door-associated electrical devices includes an adhesive- backed current carrying conduit/track that adjustably attaches to the door and transmits electrical power from the power relaying hinge to door associated electrical devices

### BACKGROUND OF THE INVENTION

### Technical Field of the Invention

The present invention relates, generally, to an easy snap-together electric power track apparatus and method for efficiently transferring electric power to doors in order to energize door associated electrical devices for the efficiently supplying electric power to doors devices such as doorknobs, door levers, door bar handles, door plates and other devices for connecting the devices with electrical energy to energize, for example, ultraviolet C sterilizing germicidal light enabled doorknobs, door levers, door bar handles and door plates and other devices that come into contact with hands so that the surfaces are, and remain, germ-free.

### Description of the Prior Art

It is well established that germs from hands are easily communicated to a person's face especially when the hands touch the face several times a minute as, for example, the eyes are rubbed, the nose is touched and other times when the hands consciously and unconsciously touch the face. Since the exposed face, more than any other part of the human body, is more susceptible to transmitting germs to the body's systems it imperative to maintain as best as possible a germ free face and hands free of pathogens which can enter the eyes, ears, mouth, nose, etc.

The relevant prior art known to the inventor includes Littles, U.S. Pat. No. 4,777,570, issued Oct. 11, 1988; Albrecht, et al., U.S. Pat. No. 10,366,094, issued Jul. 30, 2019; and Geringer et al., U.S. Patent Application Publication No. 2016/0273243, published Sep. 22, 2016.

### SUMMARY OF THE INVENTION

A primary object of the easy snap-together electric power track apparatus and method for efficiently transferring electric power to doors in order to energize door-associated electrical devices of the present invention is to provide an apparatus to safely and efficiently supply electric power to door devices in order to connect with and energize ultraviolet C sterilizing germicidal light enabled doorknobs, door levers, door bar handles, door plates, door strike plates and other door accessories.

Another object of easy snap-together electric power track apparatus and method for efficiently transferring electric power to doors in order to energize door-associated electrical devices of the present invention is to provide for the transmission of electric power to door associated devices including to recharge batteries, power lights, emergency exit lights, video displays and many other purposes with the primary purpose of the invention to efficiently transfer power to doorknobs, door levers, door bar handles, door plates, door strike plates and other door accessories for any number of uses including ultraviolet C sterilizing germicidal enabled doorknobs, door levers, door bar handles, door plates, door strike plates and other door accessories.

Another object of the easy snap-together electric power track apparatus and method for efficiently transferring electric power to doors in order to energize door-associated electrical devices of the present invention is to efficiently power ultraviolet C sterilizing germicidal light enabled door devices.

A further object of easy snap-together electric power track apparatus and method for efficiently transferring electric power to doors in order to energize door-associated electrical devices of the present invention is to provide electric power for video applications.

A still further object of the easy snap-together electric power track apparatus and method for efficiently transferring electric power to doors in order to energize door-associated electrical devices present invention to provide electric power for door enabled thru-door fans, smoke alarms and other electrical devices.

Another primary object easy snap-together electric power track apparatus and method for efficiently transferring electric power to doors in order to energize door-associated electrical devices of the present invention is to provide a low cost, safe, user friendly, easy to install wiring system to transmit electrical power to door accessories.

Another object of the easy snap-together electric power track apparatus and method for efficiently transferring electric power to doors in order to energize door-associated electrical devices the present invention is to provide a power relaying hinge between the electrical power source and the door in order to safely transmit electric power to door accessories.

Another object of the easy snap-together electric power track apparatus and method for efficiently transferring electric power to doors in order to energize door-associated electrical devices of the present invention is to provide a power relaying hinge system between the electrical power source and the door in order to safely transmit electric power to door accessories and incorporates shielded electric wires for safe use of the present invention.

Another object of the easy snap-together electric power track apparatus and method for efficiently transferring electric power to doors in order to energize door associated electrical devices of the present invention is to provide means to convert standard 120-volt and/or standard 240-volt power to low DC power for safety considerations or to insulate AC power for safe transfer to insulated doorknob should AC power be desired as opposed to DC power.

A still further object of the easy snap-together electric power track apparatus and method for efficiently transferring electric power to doors in order to energize door associated electrical devices present invention is to provide means so that the system is automatically energized.

Another object of the easy snap-together electric power track apparatus and method for efficiently transferring electric power to doors in order to energize door-associated electrical devices of the present invention is to utilize the transferred electric power for any purpose such as for observational cameras, for night and other lighting and may include energizing ultraviolet C sterilizing germicidal light enabled doorknobs, pull handles, door levers, door push plates etc, for the purpose of maintaining sterility and decontaminating contaminated surfaces from germs.

A still further object of the easy snap-together electric power track apparatus and method for efficiently transferring electric power to doors in order to energize door-associated electrical devices present invention permits the conveyed power for use with automatic locks and/or other devices.

Still another object of the easy snap-together electric power track apparatus and method for efficiently transferring electric power to doors in order to energize door-associated electrical devices present invention is to transmit electrical power from the door frame through the door latch assembly to the door.

A further object of the easy snap-together electric power track apparatus and method for efficiently transferring electric power to doors in order to energize door-associated electrical devices of the present invention is to incorporate into a doorknob, door lever and other devices ultraviolet C light so that any germs on the users hands are dissipated.

The foregoing and related objects are accomplished by the present invention, which provides a snap-together electric power track apparatus for efficiently transferring electric power to doors in order to energize door-associated electrical devices, which includes a door access device associated with a door wherein a user comes into contact with the door access device utilizing electrical energy, a power hinge for transmitting electrical power the door, and means for transmitting electrical energy to electrically energize one or more door-associated electrical devices.

Preferably, the means for transmitting electrical energy to electrically energize door-associated electrical devices includes an adhesive-backed current carrying conduit/track that adjustably attaches to the door and transmit electrical power from the power relaying hinge to door associated electrical devices and may include ultraviolet C sterilizing germicidal light enabled doorknobs, contoured door levers, rectangular door levers, door bar handles, door push plates, rechargeable batteries, lights, emergency lights video equipment and fans.

It is further preferable that when a door access device associated with the door comes into contact with a user, the door access device emits ultraviolet C that constitutes a sterilizing germicidal light, which may be provided as light emitting diodes, fiber optics, glass fibers and mercury bulbs.

In a particularly preferred embodiment, the means for transmitting electrical energy includes a power track that is either associated with the door and/or door-associated electrical devices.

Other electrical devices may also be energized by the easy snap-together electric power track apparatus and method for efficiently transferring electric power to doors in order to energize door-associated electrical devices of the present invention shown herein as, for example, existing doorknobs, door levers, door bar handles and door plates may be easily adapted and retrofitted with the adaption and incorporation of the easy snap-together power track apparatus of the present invention for the introduction of electrical power to energize ultraviolet C sterilizing germicidal light equipped devices to continuously sanitize the surface for a germ-free environment.

Ultraviolet C sterilizing germicidal light enabled doorknobs and door handles maintain a germ-free surface by disinfecting and sterilizing doorknobs, door handle levers, door bar handles, door plates and other devices that are touched by a user hands. By exposing the doorknob, door lever, door bar handle, door plates, faucet lever/handle, elevator buttons and other devices to ultraviolet C sterilizing germicidal light to kill or inactivate microorganisms by destroying nucleic acids, disrupting their DNA/RNA and leaving them unable to function or reproduce.

Other objects and features of the present invention will become apparent when considered in combination with the accompanying drawing figures, which illustrate certain preferred embodiment of the present invention. It should, however, be noted that the accompanying drawing figures are intended to illustrate only select preferred embodiments of the claimed invention and are not intended as a means for defining the limits and scope of the invention.

### BRIEF DESCRIPTION OF THE DRAWING FIGURES

In the drawing figures, wherein similar features are denoted with similar reference numerals throughout the several views:

FIG. 1 is a front view of the easy snap-together electric power track apparatus and method for efficiently transferring electric power to doors in order to energize door-associated electrical devices of the present invention as generally installed with the indicated power relaying hinge that facilitates the transmission of external electrical power to door accessories such as ultraviolet C sterilizing germicidal light-enabled and emitting doorknobs, door levers, door bar handles, door plates and door strike plates;

FIG. 2 is a partial front view of the easy snap-together electric power track apparatus and method for efficiently transferring electric power to doors in order to energize door associated electrical devices of the present invention illustrating the power relaying hinge as generally installed;

FIG. 3 is a partial, enlarged perspective view of the easy snap-together electric power track apparatus and method for efficiently transferring electric power to doors in order to energize door-associated electrical devices of the present invention supplying electrical power to an enabled ultraviolet C sterilizing germicidal light enabled doorknob;

FIG. 4 is partial perspective view of the easy snap-together electric power track apparatus and method for efficiently transferring electric power to doors in order to energize door-associated electrical devices present invention illustrating installation of the present invention;

FIG. 5 is partial perspective view of the easy snap-together electric power track apparatus and method for efficiently transferring electric power to doors in order to energize door-associated electrical devices of the present invention further illustrating installation of the invention; and,

FIG. 6 is an overview of the easy snap-together electric power track apparatus and method for efficiently transferring electric power to doors in order to energize door associated electrical devices the present invention illustrating the structure for transmitting electric current to the source of the ultraviolet C sterilizing germicidal light.

### DESCRIPTION OF THE REFERENCE NUMERALS

10 entrance and exit door

12 wall

14 hinge

16 electrical input generally 120 volts AC

18 AC/DC converter converts generally 120-volt AC to low power DC

20 power relaying hinge

22 adhesive-backed current carrying conduit/track

24 door access devices such as a ultraviolet C sterilizing germicidal light enabled doorknobs, door levers, door bar handles, door plates and other door accessories such as rechargeable batteries, lights, emergency exit lights, video equipment and fans

26 ultraviolet C sterilizing germicidal light

28 measuring tape

30 person taking measurements

32 cutting instrument

34 person cutting adhesive backed current carrying conduit/track to size

36 adhesive backing of adhesive backed electrical current carrying electrical conduit/track

38 connector

### DETAILED DESCRIPTION OF THE DRAWING FIGURES AND PREFERRED EMBODIMENTS

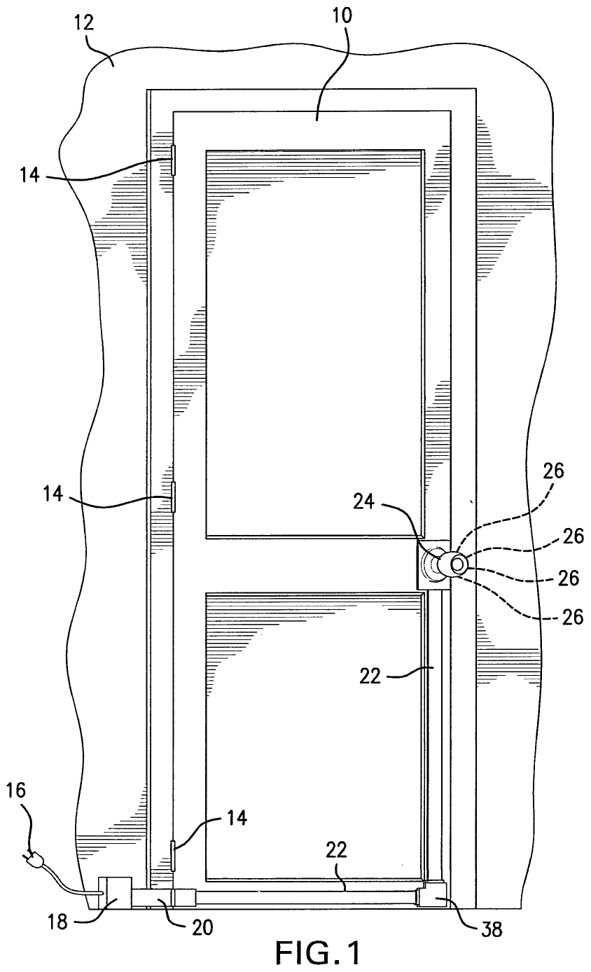

Turning now, in detail, to the accompanying drawing figures, FIG. 1 shows the structure of the easy snap-together electric power track apparatus and method for efficiently transferring electric power to doors in order to energize door-associated electrical devices the present invention to energize door associated electrical devices such as ultraviolet C sterilizing germicidal light emitting doorknob 24. Door 10 is attached to wall 12 by hinge(s) 14. Electrical input power 16 provides 120 volts AC current to AC-to-DC converter 18 resulting in low power DC current being transferred by power-relaying hinge 20 to an adhesive-backed current carrying conduit/track 22. Adhesive-backed current carrying conduit/track 22 is connected and energizes ultraviolet C sterilizing germicidal light emitting doorknob 24 which emits ultraviolet C sterilizing germicidal light 26.

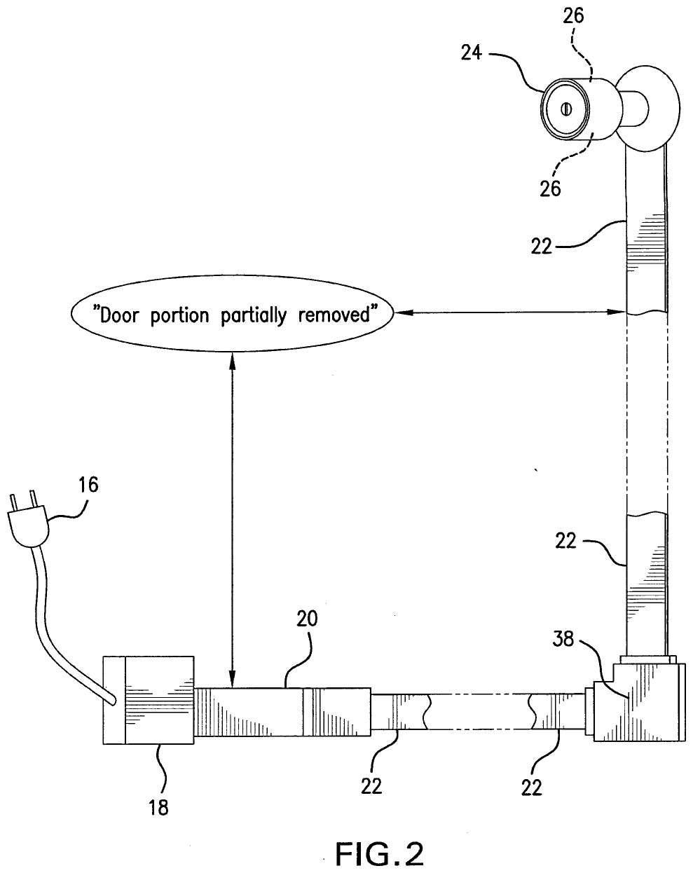

FIG. 2 is a partial front view of the easy snap-together electric power track apparatus and method for efficiently transferring electric power to doors in order to energize door-associated electrical devices the present invention illustrating the power relaying hinge 20 as generally installed on door 10. Electric power from adhesive-backed current carrying conduit/track 22 connects to door access device 24. Doorknob access device 24 has incorporated within its structure a source of ultraviolet C sterilizing germicidal light 26 so that, on contact, a person's hand is sensitized and rendered germ-free. Connector 38 provides electrical continuity of adhesive backed current carrying conduit/track 22 when a change in direction when installing to door 10 is required. Although as shown adhesive backed current carrying conduit/track 22 provides electric power to ultraviolet C sterilizing germicidal light enabled doorknob 24 the adhesive backed current carrying conduit/track 22 also powers other door-related accessories (not shown) that require transmission of electric power to door mechanisms including the recharging batteries, power lights, emergency exit lights, video displays, door levers, door bar handles, door plates, door strike plates and other door accessories that are associated with door 10. Also illustrated are components 18, 20, 22, 38 snap together to form an electrical circuit.

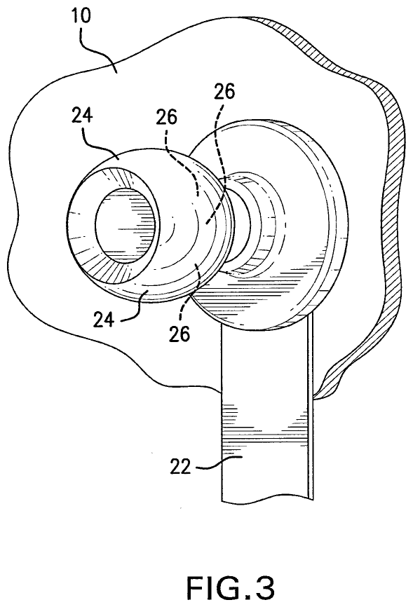

FIG. 3 shows the easy snap-together electric power track apparatus and method of the present invention for efficiently transferring electric power to doors in order to energize door associated electrical devices of the present invention supplying electrical power to a typically enabled ultraviolet C sterilizing germicidal light C enabled doorknob 24 which emits ultraviolet C sterilizing germicidal light 26. Doorknob 24 is electrically connected to adhesive backed current carrying conduit/track 22 which, in turn, is affixed to door 10.

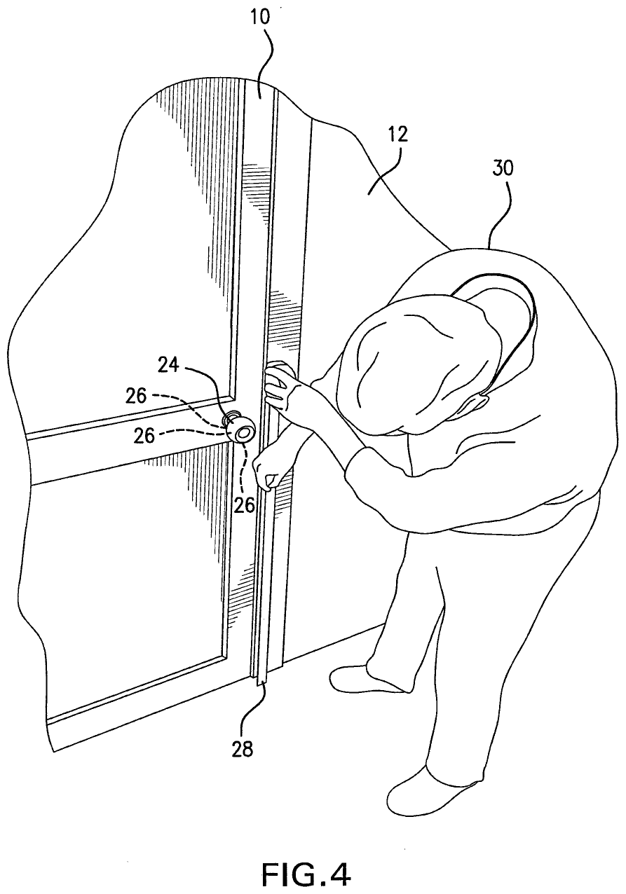

FIG. 4 is a partial perspective view of the easy snap-together electric power track apparatus and method of the present invention for efficiently transferring electric power to doors in order to energize door-associated electrical devices of the present invention and illustrating installation of the present invention. Measuring tape 28 provides for accurate installation of the invention.

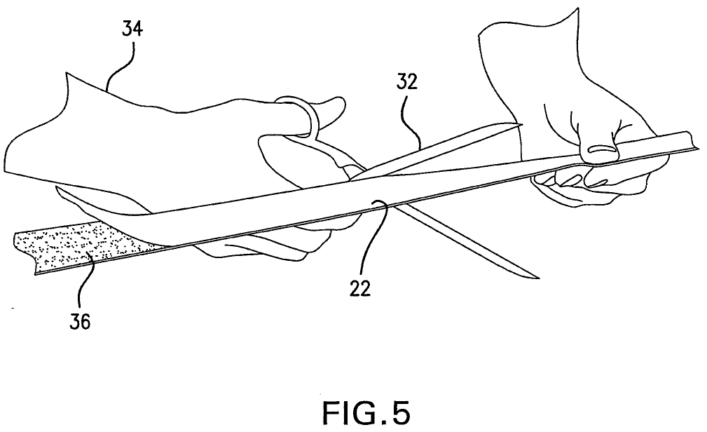

FIG. 5 is a partial perspective view of the easy snap-together electric power track apparatus and method of the present invention for efficiently transferring electric power to doors in order to energize door-associated electrical devices of the present invention further illustrating the ease of installation of the invention. Cutting instrument 32 is able to be cut to size for use with adhesive-backed current carrying conduit/track 22. Adhesive backing 36 of adhesive-backed electrical current carrying electrical conduit/track 22 can then be placed on door 10.

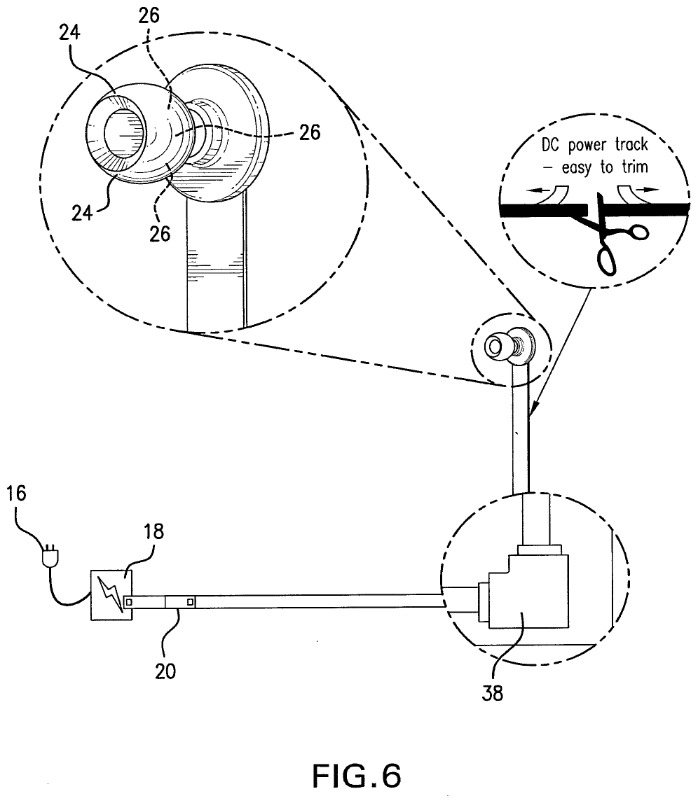

FIG. 6 is an overview of the easy snap-together electric power track apparatus and method of the present invention for efficiently transferring electric power to doors in order to energize door associated electrical devices of the present invention illustrating the structure and installation for transmitting electric current to the source of the ultraviolet C sterilizing germicidal light. Connector 38 provides for the electrical continuity of adhesive backed current carrying conduit/track 22 when installed onto door 10.

While only several embodiments of the present invention have been shown and described, it will be obvious to those skilled in the art that many modifications may be made to the present invention without departing from the spirit and scope thereof.

What is claimed is:

1. A snap-together electric power track apparatus for efficiently transferring electric power to doors for energizing door-associated electrical devices, comprising:
a door access device for a door for allowing a person to come into contact with said door access device;
a power relying hinge for the door having said door access device for transmitting electrical power from its source to the door; and,
means for transmitting electrical energy to electrically energize a door-associated electrical device.

2. The easy snap-together electric power track apparatus for efficiently transferring electric power to doors for energizing door-associated electrical devices according to claim 1, wherein said means for transmitting electrical energy to electrically energize door-associated electrical devices includes an adhesive-backed current carrying conduit or track that attaches to the door and transmits electrical power from said power relaying hinge to door-associated electrical devices.

3. The easy snap-together electric power track apparatus for efficiently transferring electric power to doors for energizing door-associated electrical devices according to claim 2, wherein said door-associated electrical devices include one or more of ultraviolet C sterilizing germicidal light enabled doorknobs, contoured door levers, rectangular door levers, door bar handles, door push plates, rechargeable batteries, lights, emergency lights video equipment and fans.

4. The easy snap-together electric power track apparatus for efficiently transferring electric power to doors for energizing door-associated electrical devices according to claim 2, wherein when said door access device with the door comes into contact with the person said door access device emits ultraviolet C sterilizing germicidal light from one or more of light emitting diodes, fiber optics, glass fibers and mercury bulbs.

5. The easy snap-together electric power track apparatus for efficiently transferring electric power to doors for energizing door-associated electrical devices according to claim 1, wherein when said door access device with the door comes into contact with the person said door access device emits ultraviolet C sterilizing germicidal light from one or more of light emitting diodes, fiber optics, glass fibers and mercury bulbs.

6. The easy snap-together electric power track apparatus for efficiently transferring electric power to doors for energizing door-associated electrical devices according to claim 1, wherein means for transmitting electrical energy to electrically energize a door-associated electrical device is via a snap-together power track to one or more of the door-associated electrical devices.
